(19) United States
(12) Patent Application Publication
(10) Pub. No.: US 2026/0154610 A1
(43) Pub. Date: Jun. 4, 2026

(54) AUDITING MACHINE LEARNING MODEL PERFORMANCE WITHOUT A TEST DATASET

(71) Applicant: GE Precision Healthcare LLC, Waukesha, WI (US)

(72) Inventors: Rahul Venkataramani, Bengaluru (IN); Chandan Kumar Mallappa Aladahalli, Bengaluru (IN)

(21) Appl. No.: 18/966,726

(22) Filed: Dec. 3, 2024

(51) Int. Cl. G06N 20/00; G16H 30/40

(52) U.S. Cl. CPC G06N 20/00; G16H 30/40

(57) ABSTRACT

Techniques for auditing machine learning (ML) model performance without a test dataset are presented. In an example, a method comprises performing, by a system comprising a processor, a model inversion attack (MIA) on a ML model and generating, by the system, synthetic input data samples corresponding to training data samples used to train the ML model based on the preforming. The method further comprises determining, by the system, one or more scope characteristics of the synthetic input data samples based on a scope assessment of the synthetic input data samples, and determining, by the system, whether the training data samples satisfy a scope criterion for the training data samples based on the one or more scope characteristics.

### TECHNICAL FIELD

This disclosure relates generally to machine learning (ML) model performance auditing, and more particularly to auditing ML model performance without a test dataset.

### BACKGROUND

Machine learning (ML) models, particularly in highly regulated industries such as healthcare, are regulated by agencies like the Food and Drug Administration (FDA) and the European Medicines Agency (EMA) to ensure patient safety, efficacy, and ethical use of data. These and similar agencies often require ML models to undergo clinical trials or equivalent validation studies to prove their safety and efficacy, similar to drugs or traditional medical devices, prior to being validated for clinical uses. In addition, regulators often require continuous auditing (or post-market surveillance) to identify any issues that might emerge once the ML model is in clinical use.

For ML models in healthcare, the regulatory validation and/or auditing process often involves assessing generalizability (performance across different populations) and robustness (reliability in different conditions) of the ML model. In this regard, regulatory frameworks increasingly include guidelines to mitigate bias and ensure fairness. Models are required to be trained on representative training datasets to prevent disparities in outputs across different patient demographic groups, conditions, and other input data sample variations that may be encountered in clinical use.

Generally, the regulatory validation and/or auditing process requires the model developer to submit materials providing a detailed description of the training dataset used to train the model, the model's architecture and the model's weights. However, due to data privacy concerns, there is no method for regulators to access and verify the training dataset used by the developer to train the model; that is, they must rely on the model developers' self-declared description of the training dataset included in the submission materials. While model performance can be verified using a test dataset, this approach requires a large and diverse data pool that is not contaminated by the training dataset. Accordingly, techniques for auditing ML model performance without a test dataset are desired.

### SUMMARY

The following presents a simplified summary of the specification in order to provide a basic understanding of some aspects of the specification. This summary is not an extensive overview of the specification. It is intended to neither identify key or critical elements of the specification, nor delineate any scope of the particular implementations of the specification or any scope of the claims. Its sole purpose is to present some concepts of the specification in a simplified form as a prelude to the more detailed description that is presented later.

According to an embodiment, a system includes at least one memory that stores computer-executable components, and at least one processor that executes the computer-executable components stored in the at least one memory. The computer-executable components can comprise an inversion component, an assessment component and a verification component. The inversion component performs a model inversion attack on a ML model, and as a result, generates synthetic input data samples corresponding to training data samples used to train the machine learning model. The assessment determines one or more scope characteristics of the synthetic input data samples based on a scope assessment of the synthetic input data samples. The verification component determines whether the training data samples satisfy a scope criterion for the training data samples based on the one or more scope characteristics.

In another embodiment, a method can comprise comprising performing, by a system comprising a processor, a model inversion attack on a ML model and generating, by the system, synthetic input data samples corresponding to training data samples used to train the machine learning model based on the preforming. The method further comprises determining, by the system, one or more scope characteristics of the synthetic input data samples based on a scope assessment of the synthetic input data samples, and determining, by the system, whether the training data samples satisfy a scope criterion for the training data samples based on the one or more scope characteristics.

In another embodiment, a non-transitory machine-readable storage medium is provide that comprises executable instructions that, when executed by a processor, facilitate performance of operations. The operations can comprise performing a model inversion attack on a machine learning model and generating synthetic input data samples corresponding to training data samples used to train the machine learning model based on the preforming. The operations further comprise determining one or more scope characteristics of the synthetic input data samples based on a scope assessment of the synthetic input data sample, and determining whether the training data samples satisfy a scope criterion for the training data samples based on the one or more scope characteristics.

### BRIEF DESCRIPTION OF THE DRAWINGS

Numerous aspects, implementations, objects and advantages of the present invention will be apparent upon consideration of the following detailed description, taken in conjunction with the accompanying drawings, in which like reference characters refer to like parts throughout, and in which:

FIG. 1 illustrates a high-level block diagram of an example computing system that facilitates auditing ML model performance without a test dataset, in accordance with one or more embodiments described herein;

FIG. 2 illustrates a high-level flow diagram of an example optimization-based model inversion process in accordance with one or more embodiments described herein;

FIG. 3 illustrates a high-level flow diagram of an example training-based model inversion process in accordance with one or more embodiments described herein;

FIG. 4 illustrates a flow diagram of an example process for verifying the scope of the training data of a ML model without a test dataset, in accordance with one or more embodiments described herein;

FIG. 5 illustrates a flow diagram of another example process for verifying the scope of the training data of a ML model without a test dataset, in accordance with one or more embodiments described herein;

FIG. 6 illustrates an example computer-implemented method for verifying the scope of the training data of a ML model without a test dataset, in accordance with one or more embodiments described herein;

FIG. 7 illustrates another example computer-implemented method for verifying the scope of the training data of a ML model without a test dataset, in accordance with one or more embodiments described herein;

FIG. 8 illustrates another example computer-implemented method for verifying the scope of the training data of a ML model without a test dataset, in accordance with one or more embodiments described herein;

FIG. 9 is a schematic block diagram illustrating a suitable operating environment; and

FIG. 10 is a schematic block diagram of a sample-computing environment.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

The disclosed subject matter is directed to systems, computer-implemented methods, apparatus and/or computer program products that facilitate auditing ML model performance without a test dataset. To facilitate this end, the disclosed techniques employ a technique known as a model inversion attack (MIA).

A MIA is a process used by malicious third parties to extract sensitive information from a ML model. The attacker uses access to the model (e.g., through an application program interface (API) or direct interaction) to infer specific attributes of the training data used to train the ML model and potentially reconstruct synthetic versions of the training data samples. In an example, an MIA has been performed on a facial recognition model to successfully reconstruct input images corresponding to those used to train the facial recognition model.

The disclosed techniques employ MIAs in novel context for good as opposed to bad. More particularly, as authorized by the ML model developer/owner, the disclosed techniques apply MIAs to ML models to facilitate assessing the generalizability and robustness of the ML models. For example, as applied to ML models subject to regulatory requirements, such as ML models used in healthcare, the disclosed techniques can be employed by regulatory agencies such as the FDA, the EMA and the like, to verify the diversity and coverage of the training dataset satisfies predefined scope criteria that ensures equitable performance across different subgroups. Customers, including hospitals and doctors, also require assurances about equitable and fair predictions from these ML models.

In this regard, in one or more embodiments, an auditing system can perform an MIA on a trained ML model to generate a synthetic input dataset that resembles the original training dataset. The synthetic input dataset is then analyzed by the auditing system to determine the scope of the synthetic input dataset in terms of diversity and coverage. For example, as applied to a ML model used in healthcare, the synthetic input dataset can be analyzed to determine different subgroups of input data samples corresponding to different patient populations (e.g., with respect to demographics, pathologies, conditions, etc.) and the number of data samples belonging to each subgroup.

The scope of the synthetic dataset can further be reported and assumed to represent the scope of the original training dataset. The scope of the synthetic dataset can also be analyzed to determine whether the scope of the synthetic dataset complies with regulatory criteria for the training dataset in terms of coverage and diversity. For example, in some implementations, the regulatory criteria can include or correspond to whether the scope of the synthetic dataset corresponds to (e.g., with respect to a defined degree of similarity) the scope of the training dataset as provided by the model developer in a training data report or the like. With these implementations, the disclosed techniques enable the auditing system to obtain synthetic data samples corresponding to snapshot of the training data samples and verify the report's veracity.

Additionally, or alternatively, the regulatory criteria can include or correspond to defined scope criteria for the training data of the ML model evaluated. For example, the defined scope criteria can include or correspond to a class diversity criterion, a class coverage criterion, a feature diversity criterion and/or a feature coverage criterion. The defined scope criteria can vary depending on the type of the ML model, the inferencing task performed by the model, a risk level associated with the model, and other factors. In some implementations, the defined scope criteria can be included in guideline information employed by the regulatory agency used to regulate usage of the ML model (e.g., the FDA, the EPA, and the like as applied to ML models used in healthcare).

In some embodiments, the synthetic dataset can also be used to infer additional information about the performance of the ML model. For example, in some implementations, the synthetic dataset can be used to infer the accuracy level of the ML model with respect to different identified subgroups of synthetic data samples based on the number of synthetic data samples included in each subgroup.

The disclosed techniques significantly enhance the transparency of the ML model, thereby fostering greater confidence in its reliability and performance. This increased transparency is achieved without compromising the privacy of the data used in the training datasets. By ensuring that the training data remains secure and confidential, the model maintains a high level of trust and integrity while providing clear insights into its decision-making processes.

In various embodiments, the disclosed techniques can be used to audit ML models used in healthcare. With these embodiments, the ML models evaluated can respectively include or correspond to a software as a medical device (SaMD). As used herein, a “SaMD” refers to refers to software intended to be used for a medical purpose. The medical purpose can include diagnosing, treating, preventing, and/or managing diseases or health conditions. In this regard, a SaMD can perform a wide range of functions, from diagnostics to treatment recommendations, and is regulated by the FDA as a medical device because of its potential impact on patient care and safety. Some examples include models configured to perform medical diagnostic tasks for disease detection, medical image analysis tasks (e.g. diagnosis, segmentation, image transformation, etc.), drug discovery tasks, medical device tasks, clinical decision support tasks, and so on.

However, the disclosed techniques are not limited to ML models corresponding to SaMDs and can be applied to other types of ML models used in the healthcare domain and other domains to analyze the scope of the training data.

The terms “algorithm” and “model” are used herein interchangeably unless context warrants particular distinction amongst the terms. The terms “artificial intelligence (AI) model” and “machine learning (ML) model” are used herein interchangeably unless context warrants particular distinction amongst the terms. Reference to an AI or ML model herein can include any type of AI or ML model, including (but not limited to): deep learning (DL) models, neural network models, deep neural network models (DNNs), convolutional neural network models (CNNs), generative adversarial neural network models (GANs), transformer models, and the like. An AI or ML model can include supervised learning models, unsupervised learning models, semi-supervised learning models, combinations thereof, and models employing other types of ML learning techniques. An AI or ML model can include a single model or a group of two or more models (e.g., an ensemble model, chained models, or the like).

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

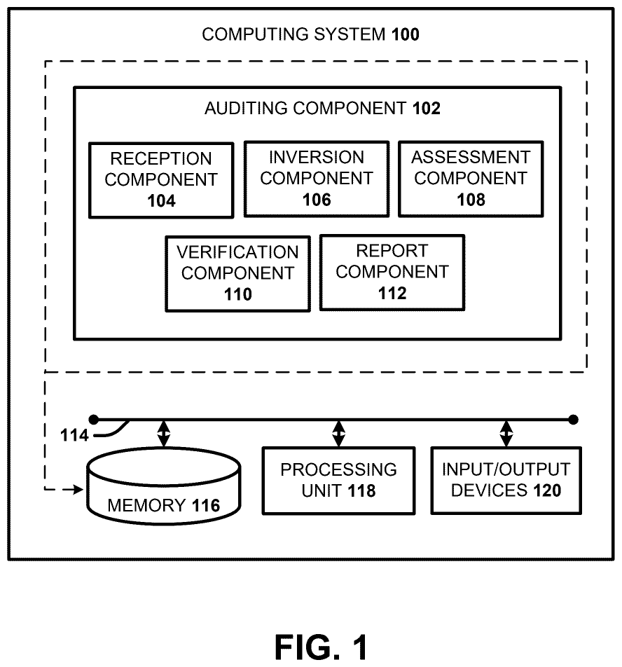

Turning now to the drawings, FIG. 1 illustrates a high-level block diagram of an example computing system 100 that facilitates auditing ML model performance without a test dataset, in accordance with one or more embodiments described herein. In some embodiments, elements described in connection with computing system 100 can be embodied in different forms such as a computer-implemented method, a computer program product, or another form. Computing system 100 can include or correspond to one or more computing devices, machines, virtual machines, computer-executable components, datastores, and the like that may communicatively coupled to one another either directly or via one or more wired or wireless communication frameworks. Aspects of the systems, apparatuses or processes explained in this disclosure can constitute computer-executable or machine-executable component(s) embodied within machine(s), e.g., embodied in one or more computer readable mediums (or media) associated with one or more machines. Such component(s), when executed by the one or more machines, e.g., computer(s), computing device(s), virtual machine(s), etc. can cause the machine(s) to perform the operations described.

In this regard, computing system 100 can comprise at least one memory 116 that stores computer-executable components, and at least one processor or processing unit 118 that executes the computer-executable components stored in the at least one memory 110. The computer-executable components comprise auditing component 102 that facilitates reviewing or auditing the performance of trained ML models with respect to generalizability and robustness without a test dataset or access to the original training dataset. To facilitate this end, the auditing component 102 can include reception component 104, inversion component 106, assessment component 108, verification component 110 and report component 112. These components respectively correspond to computer-executable components.

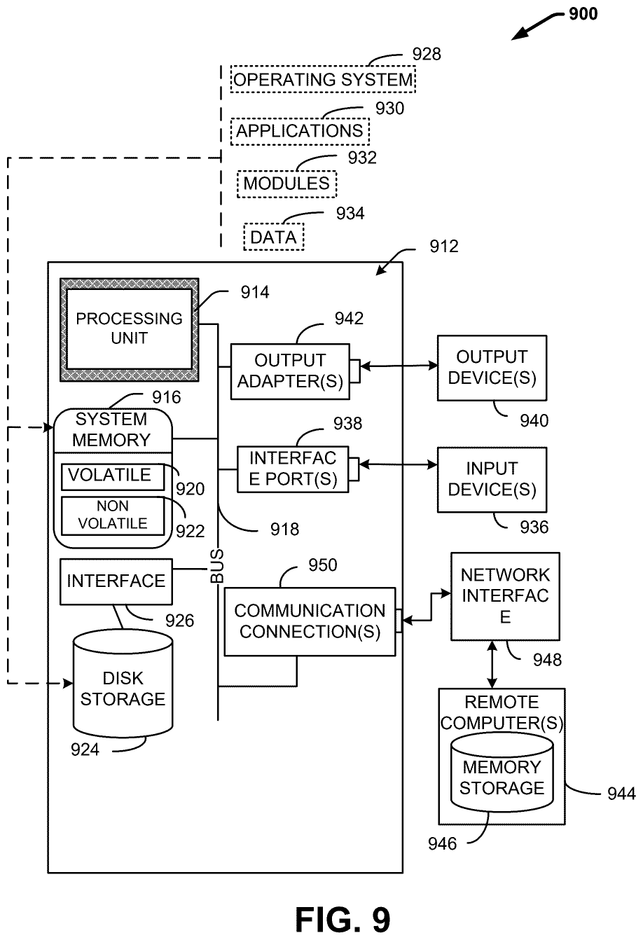

Examples of said memory 116 and processing unit 118 as well as other suitable computer or computing-based elements, can be found with reference to FIG. 9 (e.g., system memory 916 and processing unit 914 respectively), and can be used in connection with implementing one or more the components shown and described in connection with FIG. 1, or other figures disclosed herein.

Computing system 100 can further include one or more input/output devices 120 to facilitate receiving user input and rendering data (e.g., report data and the like) to users in association with performing various operations described with respect to the auditing component 102 and/or processes described herein. Suitable examples of the input/output devices 120 are described with reference to FIG. 9 (e.g., input devices 936 and output devices 940). Computing system 100 can further include a system bus 114 that couples the memory 116, the processing unit 118 and the input/output devices 120 to one another.

In various embodiments, auditing component 102 provides for auditing the performance of MLs model without a test dataset. The ML models can include or correspond to any type of trained ML model configured to perform any task. In some embodiments, the auditing component 102 may be used by a regulatory entity that regulates usage of certain types of ML models, such as ML models used for medical purposes. For example, in some implementations, the ML models can include or correspond to SaMDs that are subject to regulatory approval and performance auditing by the FDA, the EMA or the like, to ensure patient safety, accuracy, and compliance with ethical standards. With these implementations, the auditing component 102 may be configured to evaluate the performance of an ML model and generate a resulting performance report in association with reception (e.g., via reception component 104) of a submission for regulatory approval of the ML model by the model developer/owner (e.g., a 510k submission to the FDA for a SaMD or the like), and/or in accordance with a routine auditing process.

However, the disclosed techniques are not restricted to healthcare regulated ML models. In this regard, various other types of ML models are subject to regulatory restrictions. The regulation of ML models varies across jurisdictions and generally depends on their domain of application and the risks they pose to privacy, safety, fairness and social impacts. For example, financial models (e.g., credit scoring and loan approval models, fraud detection in banking, high frequency trading models, etc.) are regulated by various regulatory bodies to prevent discrimination, ensure fairness, and reduce financial risks. In another example, employment and hiring models (e.g., resume screening and candidate ranking models, predictive models for employee performance or retention) are regulated by various regulatory bodies to avoid discrimination based on protected attributes like race, gender or age. In another example, autonomous system models (e.g., autonomous vehicles and drones, robots in manufacturing or service industries) are regulated by various regulatory bodies to ensure safety, reliability and compliance with ethical guidelines.

In this regard, the auditing component 102 can be used to audit the performance of any type of ML model subject to regulatory restrictions. The regulatory process and regulatory restrictions for such ML models varies depending on the domain of application and the risks they pose to privacy, safety, fairness and social impacts. Generally, the regulatory process requires the ML model developer/owner to provide detailed information (referred to herein as the “regulatory submission information”) about the ML model to enable the regulatory entity to evaluate the ML model's performance with respect to safety, fairness, transparency and compliance standards. The specific requirements of the regulatory submission information vary based on the domain and other factors. In various embodiments, in association with evaluating the performance of an ML model without a test dataset, the auditing component 102 can receive or otherwise access (e.g., via reception component 104) the ML model itself (e.g., either directly or via an API) as well as the regulatory submission information.

Generally, the regulatory submission information includes a description of the model's intended use case, functionality, the model scope and the decision making or prediction process. The model scope information can include a definition of the range of tasks, contexts, and conditions under which the model is expected to operate. For example, the model scope information can include, but is not limited to: a definition of the task that the model is designed to do (e.g., classify, predict, recommend, etc.), a description of the types and formats of input data that the model can handle during inference, and a description of the type of predictions or decisions the model will make. The regulatory submission information also includes a description of the type of the model, the model architecture (e.g., layers, nodes, activation functions), the model weights, hyperparameters, algorithms used (e.g., loss functions), and optimization techniques.

The regulatory submission information also includes training data details, including a description of the sources of the training data, the size of the training data, and the scope of the training data. The training data scope refers to the characteristics and boundaries of the data used to train a ML model. It defines what the training data represents, its intended coverage, and its limitations. In this regard, the training data scope information can define the diversity and coverage of the training dataset. In machine learning, “diversity” and “coverage” within a training dataset refer to the extent to which the data encompasses a wide variety of examples, including different scenarios, contexts, and attributes relevant to the problem being solved, ensuring the model learns from a comprehensive and representative set of data, thus preventing bias and improving its ability to generalize to new, unseen data. Diversity represents the range of different variations within the data, including different categories, perspectives, backgrounds, and features, ensuring the model isn't just learning from a narrow set of examples. Coverage indicates how well the dataset represents the full spectrum of possible data points within the problem domain, meaning it includes enough examples from all relevant categories and subcategories. A diverse dataset helps mitigate bias by ensuring the model is exposed to a variety of data points, not just those that might be skewed towards a specific group. A well-covered dataset enables the model to make accurate predictions on new data that might have different characteristics than the training data.

The diversity of the training data can account for both class diversity and feature diversity. Class diversity and feature diversity are two distinct concepts in ML training datasets, both of which contribute to the quality and generalizability of the model. Class diversity refers to the representation and variety of all target classes (categories or labels) in the training dataset. Class diversity ensures that the training dataset includes sufficient examples for each possible output the model is expected to predict, avoiding class imbalance or exclusion. For example, let's assume the ML model is a chest X-ray diagnostic model trained to classify various different respiratory diseases (classes), such as normal (health lung images), pneumonia, tuberculosis, lung cancer, and chronic obstructive pulmonary disease (COPD). In accordance with this example, the class diversity information would identify the different classes that the ML model is trained to classify and the distribution of training data samples (X-rays in this example) with respect to the different classes, that is the number of training data samples belonging to each class. In this regard, while some diseases (like lung cancer) may naturally have fewer cases, the training dataset should include enough samples to enable the ML model to perform accurately and equitably across the different classes.

Feature diversity refers to the variety and distribution of the attributes or characteristics (features) present in the training dataset, regardless of the class labels. For example, for a healthcare dataset, the features of the training data samples can be diverse as a function of different patient features of respective patients represented by the training data samples. The different patient features can reflect various relevant attributes about the patients, including demographic attributes (e.g., age, gender, ethnicity, location) and clinical attributes (e.g., medical conditions, comorbidities, medications, medical histories, etc.). For healthcare datasets, the features are not limited to attributes of the patients themselves and can cover a wide range of relevant features. For example, as applied to medical images, feature diversity can account for images captured by different medical imaging devices and/or under different acquisition protocols or parameters. In this regard, class diversity ensures the model learns to predict all possible outcomes, while feature diversity ensures the model understands the wide range of input conditions leading to those outcomes.

In this regard, in some embodiments, in association with evaluating the performance of an ML model, the reception component 104 can receive or otherwise access (e.g., in memory 116 and/or at an external source accessible via any suitable wired or wireless communication technology) training data report information (e.g., included in the regulatory submission information) defining the scope of the original training dataset in detail. For example, the training data report information can define the class distribution of the training data samples (e.g., the different classes and the number of samples belonging to each class). The training report information can also define the relevant feature diversity aspects of the training data samples. For example, the feature diversity information can identify different groups of training data samples corresponding to different features or feature combinations and the distributions of the training data samples amongst the different groups (e.g., number of training data samples belonging to each group).

Of particular importance, the disclosed techniques are used to assess the performance of an ML model without access to the original training dataset and without a test dataset. This enables the privacy and security of the original training dataset to be maintained and mitigates privacy concerns related to sharing of the original training data with third parties. To facilitate this end, the inversion component 106 performs a performs a model inversion attack (MIA) on the ML model, and as a result, generates synthetic input data samples corresponding to training data samples used to train the ML model. The assessment component 108 further evaluates the synthetic data samples to determine one or more scope characteristics of the synthetic input data samples based on a scope assessment of the synthetic input data samples, and the verification component 110 determines whether the training data samples satisfy a scope criterion for the training data samples based on the one or more characteristics.

In this regard, the one or more scope characteristics can include one or more distributions of the synthetic input data samples, including a class distribution and one or more feature distributions. For example, the class distribution can identify the number of different synthetic data samples belonging to each class that the ML model is asserted to detect (as provided in the training data report information), such as different chest diseases present in X-ray images of the chest. The one or more feature distributions can identify the number of different synthetic data samples respectively belonging to different feature groups. In some embodiments as applied to healthcare data, the different feature groups can account for different patient features, such as different demographic features (e.g., age, gender, ethnicity, location, etc.). For example, one feature distribution may identify the number of synthetic data samples belonging to different defined age groups, another feature distribution may identify the number of synthetic data samples belonging to different ethnicity groups, etc. In another example, as applied to medical images, the different feature distributions may include a feature distribution that identifies the number of synthetic data samples belonging to different feature groups corresponding to different acquisition parameters/protocols.

To this end, the scope assessment performed by the assessment component 108 involves determining scope information describing the one or more scope characteristics (e.g., a class distribution and one or more feature distributions) of the synthetic data samples. The verification component 110 further compares the scope information to the scope criterion (or criteria) to determine whether the scope of the synthetic data samples satisfy the scope criterion. In this regard, the scope criterion can correspond to a required, preferred or declared (e.g., in the training report data) class distribution (referred to herein as a target class distribution), and/or a required preferred or declared (e.g., in the training report data) feature distribution (referred to herein as a target feature distribution). For example, the target class distribution can indicate an acceptable amount (e.g., a number or percentage) of data samples belonging to each class and an acceptable degree of deviation between the amount of data samples belonging to different classes. Likewise, a target feature distribution can indicate an acceptable amount (e.g., a number or percentage) of data samples belonging to each feature groups of amounts different feature groups and an acceptable degree of deviation between the amount of data samples belonging to the different feature groups.

In some embodiments, information defining the target class and/or target feature distributions can be declared in the training report data. Additionally, or alternatively, the scope criterion evaluated, that is the target class and/or feature distributions, can include or correspond to a regulatory criterion. For example, as applied to ML models used in healthcare and subject to regulatory approval by the FDA as SaMD, the FDA may require that the ML model demonstrate equitable performance across different patient groups corresponding to different demographic features (e.g., different age groups, different genders, different ethnicities, etc.). In accordance with this example, the regulatory criterion can indicate the acceptable amount of training data samples that belong to the different patient demographic groups (to ensure each group is adequately covered) and an acceptable degree of deviation between the amount of data samples belonging to the different patient groups (to ensure the groups are balanced). In this regard, the regulatory criterion can include or correspond to a scope criterion required or preferred for the training data samples of the ML model by a regulatory agency that regulates usage of the ML model. The regulatory criterion can be predefined and stored in memory 116 or accessible to the auditing component 102 at a network accessible source.

The reporting component 112 can further generate a performance report comprising or summarizing the results of the assessment component 108 and/or the verification component 110 which can be stored (e.g., in memory 116), rendered (e.g., via one or more input/output devices 120), provided back to the model developer/owner, provided to the model consumers/clients, or the like.

In this regard, a MIA is a process used by malicious third parties to extract sensitive information from a ML model. The attacker uses access to the model (e.g., through an API or direct interaction) to infer specific attributes of the training data used to train the ML model and potentially reconstruct synthetic versions of the training data samples. In an example, an MIA has been performed on a facial recognition model to successfully reconstruct input images corresponding to those used to train the facial recognition model. On the contrary, the disclosed techniques employ MIAs in novel context for good as opposed to bad. More particularly, as authorized by the ML model developer/owner, the inversion component 106 employs an MIA (also referred to herein as a “model inversion process”, “inversion process,” or the like) to generate a synthetic dataset that corresponds to the original training dataset used to train the ML model.

There have been many research efforts in model inversion, which aims to obtain information about the training data from the model's predictions. In this regard, various types of model inversion attacks have been developed, including reverse engineering-based approaches, membership inference approaches, optimization methods, training-based methods, and others. In this regard, the particular inversion process used by the inversion component 106 can vary and include various existing and future developed MIA processes.

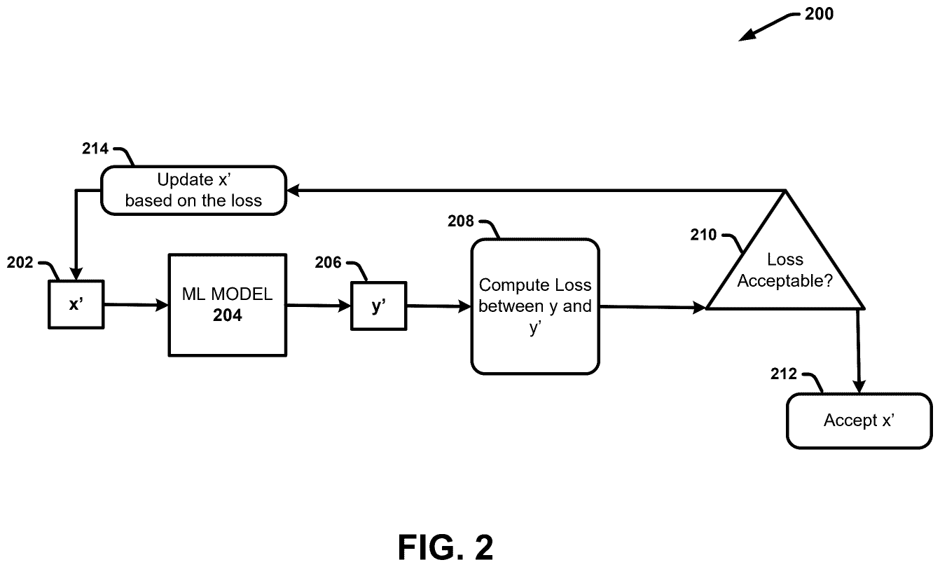

FIG. 2 illustrates a high-level flow diagram of an example optimization-based model inversion process 200 (hereinafter process 200) in accordance with one or more embodiments described herein. In various embodiments, the inversion component 106 can employ process 200 to generate synthetic data samples corresponding to the original training data samples used to train an ML model.

Optimization methods are techniques used in MIAs to manipulate the outputs of a ML model by iteratively refining input data. The attackers employ optimization algorithms or gradients to generate input data that maximizes the likelihood of a desired outcome from the model, such as inferring sensitive information about the training data or individual data points.

In this regard, as illustrated in FIG. 2, process 200 starts at 202 with feeding the ML model 204 (which corresponds to any trained ML model) an initial input data sample x′. The initial input data sample x′ can be tailored or selected based on training data report information (or the like) describing the details of the training dataset, including the type of the input data samples and the features and class labels of the training data samples. In other implementation, the initial training data sample x′ can include noise data. Let's assume by way of example that the ML model 204 corresponds to the SaMD configured to classify the different diseases in the chest X-rays described above. In accordance with this example, the initial input data sample x′ can include noise X-ray data, and/or an X-ray image of the chest with noise injected. In some embodiments, the initial data sample x′ (and additional initial data samples) can be stored in memory 116 (or another memory device accessible to the inversion component 106). In other embodiments, the inversion component 106 can generate the initial data samples used in process 200 based on the training data report information.

At 206, the ML model 204 generates an output y′ based on processing the initial data sample x′. At 208, the loss between the desired output y and y′ is computed using an applicable loss function. In this regard the desired output y corresponds to a known or desired output to be generated by the ML model as provided in the regulatory submission information. For example, as applied to the chest X-ray model, y can correspond to one of the selected classification respiratory disease outputs that the model is configured to perform (e.g., normal, pneumonia, tuberculosis, lung and COPD). It should be appreciated that in accordance with this example, process 200 can be performed repeatedly for each of the different classifications (e.g., using a different value for y). At 210, the loss is compared to acceptable loss criteria to determine whether the loss is acceptable. Assuming this is the first past through the model, the loss generally will be unacceptable. In this regard, based on the loss being unacceptable at 210, process 200 continues to 214, wherein x′ is updated based on the loss and the updated x′ is then processed back through the ML model 204 to generate a new y′ and to compute a new loss between y and the new y′ which is then evaluated again at 210. If the loss is acceptable at 210, then at 212 the system accepts x′ as being a valid input. In other words, process 200 is an iterative process that repeats until the loss is acceptable at 210 (or convergence is reached and/or a defined number of iterations have been performed), at which time the final x′ is accepted as complete at 212. The resulting x′ generated using process 200 is a synthetic data sample that corresponds to a training data sample used to train the ML model 204. For example, as applied to the chest X-ray model, x′ would be a reconstructed or synthetic X-ray image that depicts the target disease classification. In this regard, the updating of x′ at 214 involves changing x′ (e.g., modifying the x-ray) which is performed iteratively until x′ results in an acceptable loss between y and y′.

More formally, the optimization-based inversion process (e.g., process 200) can be described as follows:

Consider a model (fθ) trained on dataset

$$S = {\{ {x}_{i} , {y}_{i} \}}_{1}^{N} .$$

The model inversion attack poses reconstructing the input \hat{x} for a given output y in the form of an optimization problem in accordance with Equations 1 and 2 below. For a known output y, the target input image (e.g., the final version of x′ resulting from process 200) can be denoted as \hat{x} and is defined in accordance with Equation 1, where L(:, :) is a loss function between prediction of x′ and y, and wherein \hat{x} is obtained through gradient descent in accordance with Equation 2.

$$\hat{x} = argmin \text{?} L ( {f}_{θ} ( {x}^{′} ) , y ) . \qquad Equation 1$$

$$\hat{x} ( t + 1 ) = \hat{x} ( t ) - η ∇ {x}^{′} \vert f ( {x}^{′} ) - y \vert . \qquad Equation 2$$

$$\text{?} \text{indicates text missing or illegible when filed}$$

In this regard, process 200 is an iterative process that is performed repeatedly to generate a single synthetic data sample \hat{x}. It should be appreciated that the inversion component 106 can perform process 200 a plurality of times to generate a synesthetic dataset comprising a plurality of different synthetic data samples.

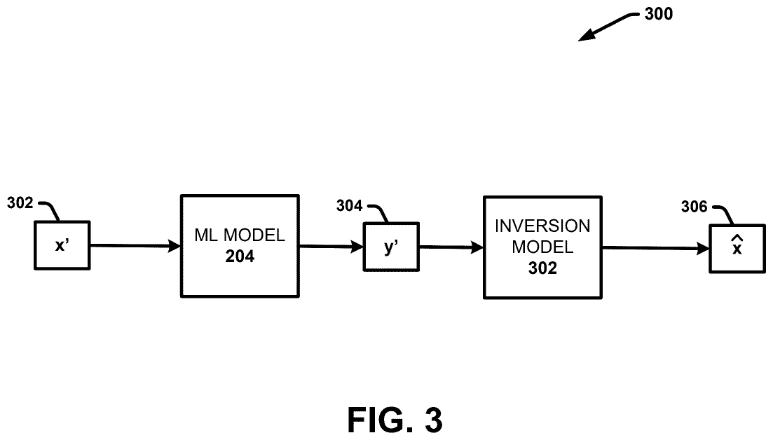

FIG. 3 illustrates a high-level flow diagram of an example training-based model inversion process 300 (hereinafter process 300) in accordance with one or more embodiments described herein. In various embodiments, the inversion component 106 can employ process 300 to generate synthetic data samples corresponding to the original training data samples used to train an ML model.

Unlike the optimization-based approach that invert the model output directly, here and inversion model 302 is first trained, which later takes the output of the ML model 204 as input and output the synthetic, reconstructed input data sample \hat{x}. This is similar to an autoencoder, where the ML model 204 behaves like the encoder and the inversion model behaves like the decoder. In this regard, as shown in FIG. 3, at 202, the inversion component 106 supplies a generic input data sample or noise x′ as input to the ML model 204 which generates an output y′ as 304. The output y′ is then supplied as input to the trained inversion model 302 which inverts the output y′ to generate the optimal version of x′ at 306, which is considered a final synthetic data sample). With these embodiments, the inversion component 106 can train the inversion model 302 using the ML model 204 and a non-trained version of the inversion model 302.

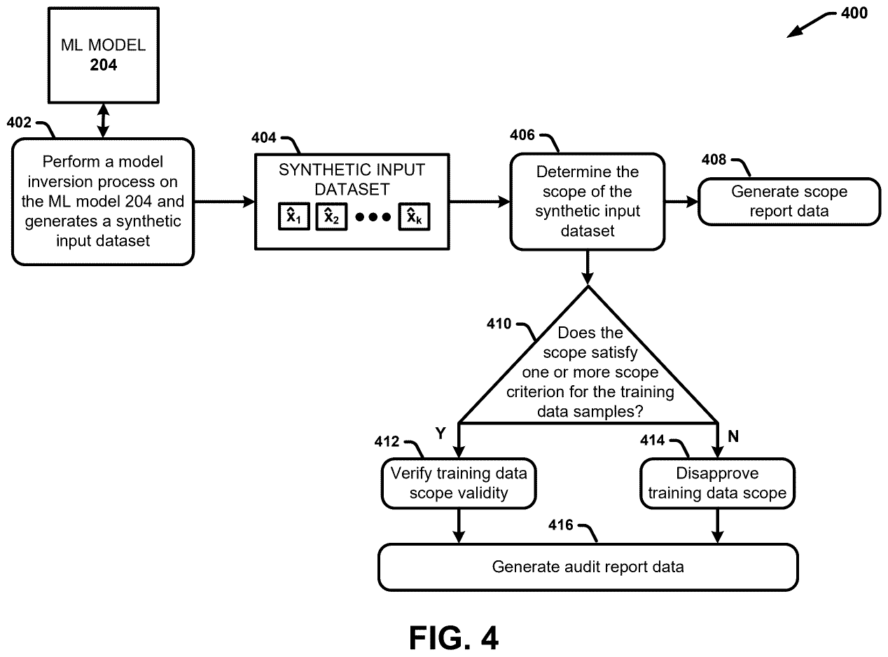

FIG. 4 illustrates a flow diagram of an example process 400 for verifying the scope of the training data of a ML model without a test dataset, in accordance with one or more embodiments described herein. With reference to FIG. 4 in view of FIGS. 1-3, in accordance with process 400, at 402, the inversion component performs a model inversion process (e.g., process 200, process 300, or another type of model inversion process) on the ML model 204 and generates a synthetic input dataset 404. The synthetic input dataset 404 comprises a plurality of synthetic input data samples (e.g., x₁-xₖ, wherein the number k can vary), and resembles the original training dataset used to train the ML model 204.

At 406, the assessment component 108 determines the scope of the synthetic input dataset 406 which is assumed to represent the scope of the original training dataset. The scope of the synthetic data set can reflect class diversity, class coverage, feature diversity and/or feature coverage. For example, in various embodiments, the scope of the synthetic dataset can reflect the class distribution of the synthetic input dataset 404, and/or one or more feature distributions. In this regard, in association with determining the scope of the synthetic input dataset at 406, the assessment component 108 can evaluate the synthetic input data samples with respect to the different output data classes (where applicable) and identify the number of data samples belonging to each class (i.e., the class distribution and coverage). To this end, the output data classes can be identified and defined in the training report data provided by the ML model developer/owner. Likewise, the assessment component 108 can group the synthetic data samples into different groups based on relevant features to determine the corresponding feature distributions. Such relevant features can be predefined (e.g., as provided in the training data report data) and/or predefined by regulatory guidelines governing the type of the ML model, the risk level, and other factors. For example, in some implementations, the training report data can identify relevant groupings of the training data samples, wherein the groupings are based on defined relevant features. Additionally, or alternatively, the relevant groupings can be based on one or more regulatory agency-based diversity requirements for the training data of the ML model 204 as defined based on the type and risk level of the ML model (e.g., as defined in applicable regulatory guidelines).

For example, as applied to SaMDs, in some embodiments, the relevant features can include or correspond to patient demographic features. In accordance with this example, different groups of the synthetic data samples can respectively correspond to different patient groups respectively corresponding to different demographic groups (e.g., with respect to age, gender, ethnicity, location, etc.). In another example, as applied to SaMDs and different patient groups, the different patient groups can reflect relevant clinical features such that the different patient groups reflect different data samples belonging to patients with different clinical features (e.g., pathologies, conditions, medical histories, etc.). In another example, as applied to SaMDs configured to process medical images, in some embodiments, the relevant features can include acquisition parameters, and the different groups can correspond to different acquisition parameters. In some embodiments, information defining different relevant feature groupings of the training data samples to be applied at 406 can be extracted from the training report data as described with respect to the scope of the original training data.

At 408, the reporting component 112 can generate scope report data describing the scope of the synthetic input dataset 404. For example, the scope report data can identify the class distribution and/or one or more feature distributions of the synthetic data samples. To this end, the respective distributions can identify the respective classes and/or features for which the respective distributions are based, the different categorical value or values applied to distinguish between each group within a distribution, and the number of data samples belonging to each group. In some embodiments, in association with generating the scope report data, the assessment component 108 can also infer an expected accuracy level of the ML model 204 with respect to the different classes and/or the different feature groups of the synthetic data samples based on the number of synthetic data samples belonging to each group. For example, let's assume the synthetic data samples are grouped and evaluated with respect to patient age such that the groups include three different groupings, including a first grouping of patients under 30, a second grouping of patients between 30 and 50, and third group of patients over 50. Based on the number of synthetic data samples in each group being the same or substantially the same, the assessment component 110 can infer that the ML model 204 performs fairly or equally across the different age groups. The scope report data can be stored (e.g., in memory 116), rendered, sent to another system of device, or the like.

At 410, the verification component 112 determines whether the scope satisfies one or more scope criterion for the training data samples. In various embodiments, the scope criterion can include or correspond to a regulatory criterion, such as a preferred or required diversity and/or coverage requirement for the training dataset applied by a regulatory body that controls approval of the ML model 204 for usage. For example, the one or more defined scope criterion can include or correspond to one or more target distributions of the synthetic input data samples with respect to one or more target features and/or classes. For instance, assuming the distributions of the synthetic data samples include a distribution of data samples belonging to different patient groups corresponding to different demographic profiles (e.g., grouped by age, gender, ethnicity, location, and the like) or another patient-based feature that can vary amongst the training data samples, the one or more scope criterion for the training data samples can include an acceptable measure of deviation between the distribution and a target distribution for the different groups. In accordance with this example, the analysis at 410 can include comparing the distribution to the target distribution and determining whether the measure of deviation between the distribution and the target distribution is acceptable. Generally, this corresponds to determining whether the ML model 204 performs fairly or substantially equally across different relevant patient groups and other feature and class groupings. In this regard, the target distributions can be tailored to ensure equitable performance across the different patient groups and other feature and class groupings.

If at 410, the verification component 110 determines that the scope of the synthetic input dataset 404 satisfies the one or more scope criterion, then at 412, the verification component can verify the training data scope validity. In other words, the verification component 110 can verify that the training data scope satisfies a diversity requirement and a coverage requirement with respect to one or more defined features and classes. However, if at 410 the verification component 110 determines that the scope of the synthetic input dataset does not satisfy the one or more scope criterion, then at 414, the verification component can disapprove the training data scope. In other words, the verification component can verify that the training data scope does not satisfy a diversity requirement and/or a coverage requirement with respect to defined features and classes.

At 416, the reporting component 112 can further generate audit report data that comprises or summarizes the results of the assessment component 108 and/or the verification component 110. The level of granularity of the audit report data can vary. For example, in some embodiments, the audit report data can include the scope report data as well as assessment data that indicates any identified disparities (and agreements) between the scope report data and the one or more scope criterion. In another example, the audit report data can simply indicate that the training data scope is either verified as acceptable or not acceptable. The reporting component 112 can store the audit report data, render the audit report data, and/or send the audit report data to an external device or system. In this regard, in various embodiments in which computing system 100 is employed by a third-party regulatory entity to audit the performance of the ML model 204, the reporting component 112 can provide the audit report data back to the model developer/owner (e.g., using any suitable electronic reporting technology). In addition, consumers of the ML model 204 (e.g., customers, including hospitals, doctors, patients, etc. as applied to the ML model 204 being a SaMD), also require assurances about equitable and fair predictions from the ML model 204. Thus, the reporting component 112 can also provide the audit report data to any authorized entity that is interested in understanding the performance of the model as a function of the scope of the training data.

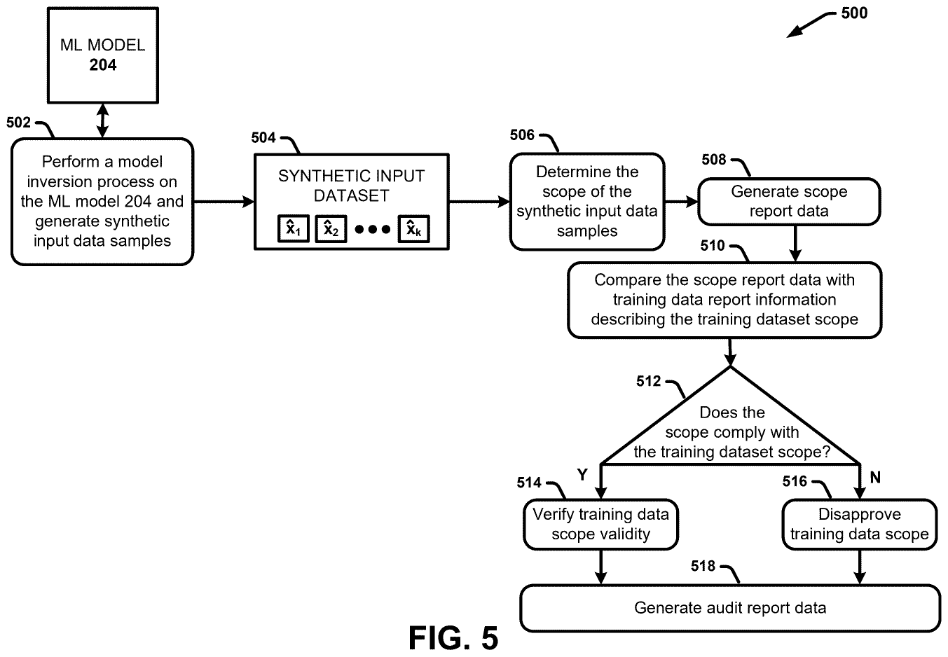

FIG. 5 illustrates a flow diagram of another example process 500 for verifying the scope of the training data of a ML model without a test dataset, in accordance with one or more embodiments described herein. Process 500 is similar to process 400 yet with some minor variations noted below. Repetitive description of like elements in respective embodiments is omitted for sake of brevity. With reference to FIG. 5 in view of FIGS. 1-4, in accordance with process 500, at 502, the inversion component performs a model inversion process (e.g., process 200, process 300, or another type of model inversion process) on the ML model 204 and generates a synthetic input dataset 504. The synthetic input dataset 504 comprises a plurality of synthetic input data samples (e.g., x₁-xₖ, wherein the number k can vary), and resembles the original training dataset used to train the ML model 204.

At 506, the assessment component 108 determines the scope of the synthetic input dataset 406 which is assumed to represent the scope of the original training dataset, as described with reference to 406 of process 400. At 508, the reporting component 112 can generate scope report data describing the scope of the synthetic input dataset 504, as described with reference to 408 of process 400.

At 510, the verification component 110 compares the scope report data with training report information describing the training data set scope. For example, the scope report data can identify the class distribution and/or one or more feature distributions of the synthetic data samples, and the training report information can include one or predefined class and/or feature distributions of the training dataset (e.g., as declared by the model developer/owner in the training report data submitted). To this end, the respective distributions can identify the respective classes and/or features for which the respective distributions are based, the different categorical value or values applied to distinguish between each group within a distribution, and the number of data samples belonging to each group. In association with comparing the scope report data with the training report information at 510, the verification component 110 can perform differential analysis between the two datasets to identify any measures of disparity between the respective datasets.

At 512, the verification component 110 determines whether the scope of the synthetic dataset complies with the scope of the training dataset as described in the training report information. This can be based on whether any measure of disparity were identified, the number of disparities, the degree of the disparities, and defined acceptable thresholds for such disparities with respect to the number and/or degree.

If at 512 the verification component 110 determines that the scope of the synthetic input dataset 504 complies with the training dataset scope, then at 514, the verification component 110 can verify the training data scope validity. In other words, the verification component 110 can verify that the training dataset scope as declared by the model developer/owner in the training data report information is accurate. However, if at 512 the verification component 110 determines that the scope of the synthetic input dataset does not satisfy the training dataset scope, then at 516, the verification component 110 can disapprove the training data scope. In other words, the verification component 110 can verify that the training dataset scope as declared by the model developer/owner in the training data report information is inaccurate.

At 518, the reporting component 112 can further generate audit report data that comprises or summarizes the results of the assessment component 108 and/or the verification component 110. The level of granularity of the audit report data can vary. For example, in some embodiments, the audit report data can include the scope report data as well as assessment data that indicates whether the training dataset scope is verified or not and the basis for the decision. In another example, the audit report data can simply indicate that the training data scope is either verified as acceptable or not acceptable. The reporting component 112 can store the audit report data, render the audit report data, and/or send the audit report data to an external device or system. In this regard, in various embodiments in which computing system 100 is employed by a third-party regulatory entity to audit the performance of the ML model 204, the reporting component 112 can provide the audit report data back to the model developer/owner (e.g., using any suitable electronic reporting technology). In addition, consumers of the ML model 204 (e.g., customers, including hospitals, doctors, patients, etc. as applied to the ML model 204 being a SaMD), also require assurances about equitable and fair predictions from the ML model 204. Thus, the reporting component 112 can also provide the audit report data to any authorized entity that is interested in understanding the performance of the model as a function of the scope of the training data.

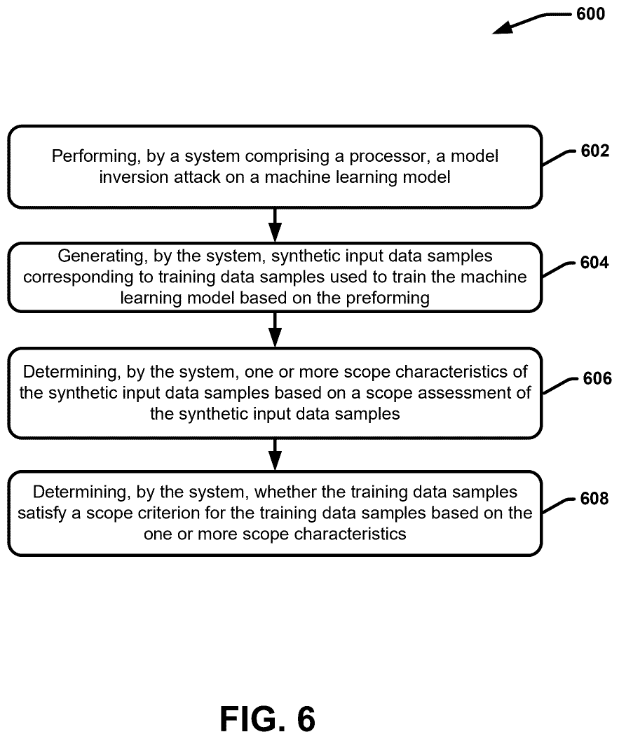

FIG. 6 illustrates an example computer-implemented method 600 for verifying the scope of the training data of a ML model without a test dataset, in accordance with one or more embodiments described herein. With reference to FIG. 6 in view of FIGS. 1-5, method 600 corresponds to an example method that can be performed by computing system 100 in accordance with an embodiment. At 602, method 600 comprises performing, by a system comprising a processor (e.g., computing system 100), a model inversion attack (MIA) on a machine learning (ML) model (e.g., using inversion component 106). At 604, method 600 comprises generating, by the system, synthetic input data samples corresponding to training data samples used to train the ML model based on the preforming (e.g., via inversion component 106).

At 606, method 600 comprises determining, by the system, one or more scope characteristics of the synthetic input data samples based on a scope assessment of the synthetic input data samples (e.g., via assessment component 108). For example, the one or more scope characteristics can account for the diversity and coverage of the synthetic input data samples with respect to one or more features and/or classes. In this regard, the one or more scope characteristics can include corresponding class and/or feature distributions of the synthetic input data samples. At 608, method 600 comprises determining, by the system, whether the training data samples satisfy a scope criterion for the training data samples based on the one or more characteristics (e.g., via verification component 110).

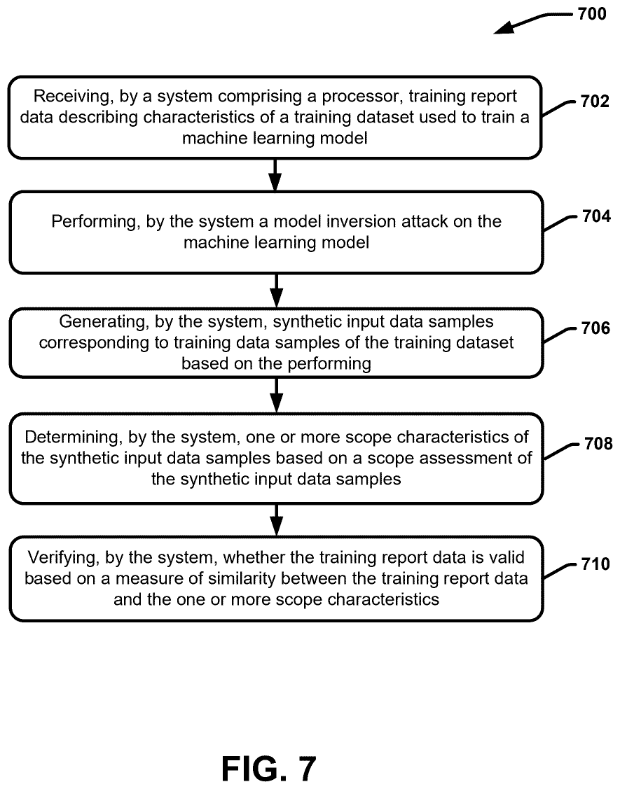

FIG. 7 illustrates another example computer-implemented method 700 for verifying the scope of the training data of a ML model without a test dataset, in accordance with one or more embodiments described herein. With reference to FIG. 7 in view of FIGS. 1-5, method 700 corresponds to an example method that can be performed by computing system 100 in accordance with an embodiment. At 702, a system comprising a processor (e.g., computing system 100), receives training report data describing characteristics of a training dataset used to train a ML model. At 704, method 700 comprises performing, by the system, a model inversion attack (MIA) on the ML model (e.g., using inversion component 106). At 706, method 700 comprises generating, by the system, synthetic input data samples corresponding to training data samples of the training dataset based on the preforming (e.g., via inversion component 106). At 708, method 700 comprises determining, by the system, one or more scope characteristics of the synthetic input data samples based on a scope assessment of the synthetic input data samples (e.g., via assessment component 108). At 710, method 700 comprises verifying, by the system, whether the training report data is valid based on a measure of similarity between the training report data and the one or more scope characteristics (e.g., via verification component 110).

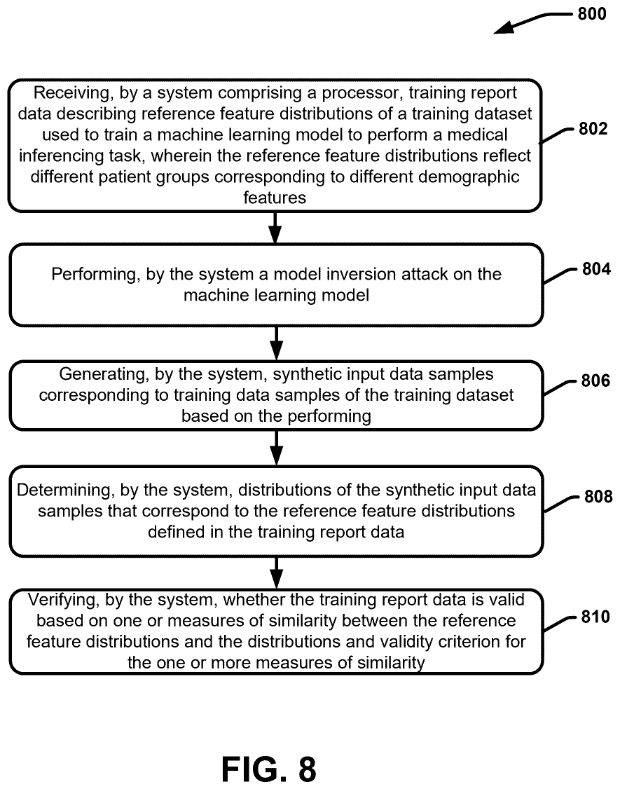

FIG. 8 illustrates another example computer-implemented method 800 for verifying the scope of the training data of a ML model without a test dataset, in accordance with one or more embodiments described herein. With reference to FIG. 8 in view of FIGS. 1-5, method 800 corresponds to an example method that can be performed by computing system 100 in accordance with an embodiment. At 802, a system comprising a processor (e.g., computing system 100), receives (e.g., via reception component 104) training report data describing reference feature distributions of a training dataset used to train a ML model to perform a medical inferencing task (e.g., disease diagnosis, medical image analysis, or any other type of medical inferencing task that constitutes the ML model as being classified as a SaMD), wherein the reference feature distributions reflet different patient groups corresponding to different demographic features. In other words, the training report data can include information that identifies the number of training data samples belonging to different demographic feature categories or groupings of the training data samples, such as the number of training data samples belonging to different genders, age groups, races, locations, etc.

At 804, method 800 comprises performing, by the system, a model inversion attack (MIA) on the ML model (e.g., using inversion component 106). At 806, method 800 comprises generating, by the system, synthetic input data samples corresponding to training data samples of the training dataset based on the preforming (e.g., via inversion component 106). At 808, method 800 comprises determining, by the system, distributions of the synthetic input data samples that correspond to the reference feature distributions defined in the training report data (e.g., via assessment component 108). At 810, method 800 comprises verifying, by the system, whether the training report data is valid based one or more measure of similarity between the reference feature distributions and the distributions and validity criterion (e.g., a minimum level of similarity threshold or the like) for the one or more measures of similarity (e.g., via verification component 110).

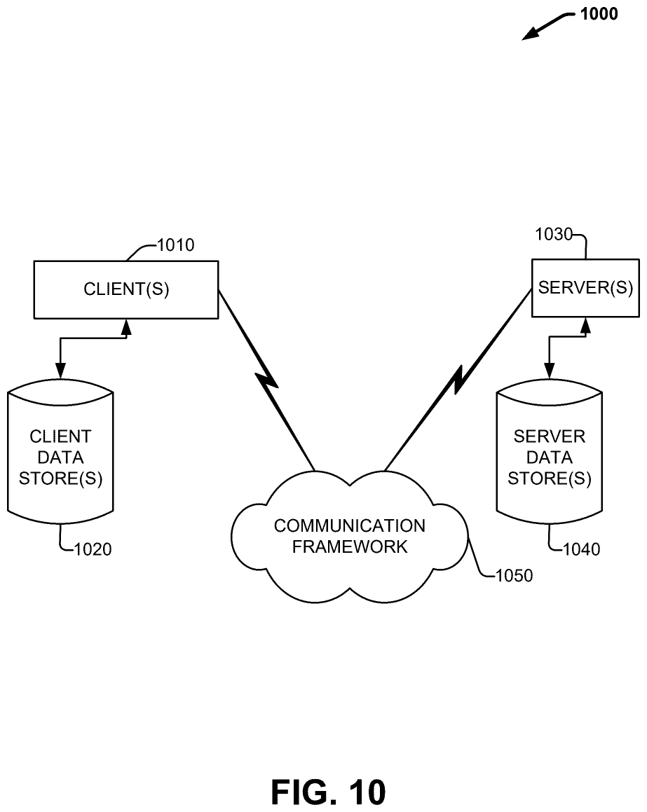

The subject matter, FIGS. 9 and 10 as well as the following discussion are intended to provide a brief, general description of a suitable environment in which the various aspects of the disclosed subject matter may be implemented.

With reference to FIG. 9, a suitable environment 900 for implementing various aspects of this disclosure includes a computer 912. The computer 912 includes a processing unit 914, a system memory 916, and a system bus 918. The system bus 918 couples system components including, but not limited to, the system memory 916 to the processing unit 914. The processing unit 914 can be any of various available processors. Dual microprocessors and other multiprocessor architectures also can be employed as the processing unit 914.

The system bus 918 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 916 includes volatile memory 920 and nonvolatile memory 922. The basic input/output system (BIOS), containing the basic routines to transfer information between elements within the computer 912, such as during start-up, is stored in nonvolatile memory 922. By way of illustration, and not limitation, nonvolatile memory 922 can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory 920 includes random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 912 also includes removable/non-removable, volatile/non-volatile computer storage media. FIG. 9 illustrates, for example, a disk storage 924. Disk storage 924 includes, but is not limited to, devices like a magnetic disk drive, floppy disk drive, tape drive, Jaz drive, Zip drive, LS-90 drive, flash memory card, or memory stick. The disk storage 924 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 924 to the system bus 918, a removable or non-removable interface is typically used, such as interface 926.

FIG. 9 also depicts software that acts as an intermediary between users and the basic computer resources described in the suitable operating environment 900. Such software includes, for example, an operating system 928. Operating system 928, which can be stored on disk storage 924, acts to control and allocate resources of the computer system 912. System applications 930 take advantage of the management of resources by operating system 928 through program modules 932 and program data 934, e.g., stored either in system memory 916 or on disk storage 924. It is to be appreciated that this disclosure can be implemented with various operating systems or combinations of operating systems.

A user enters commands or information into the computer 912 through input device(s) 936. Input devices 936 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 914 through the system bus 918 via interface port(s) 938. Interface port(s) 938 include, for example, a serial port, a parallel port, a game port, and a universal serial bus (USB). Output device(s) 940 use some of the same type of ports as input device(s) 936. Thus, for example, a USB port may be used to provide input to computer 912, and to output information from computer 912 to an output device 940. Output adapter 942 is provided to illustrate that there are some output devices 940 like monitors, speakers, and printers, among other output devices 940, which require special adapters. The output adapters 942 include, by way of illustration and not limitation, video and sound cards that provide a means of connection between the output device 940 and the system bus 918. It should be noted that other devices and/or systems of devices provide both input and output capabilities such as remote computer(s) 944.

Computer 912 can operate in a networked environment using logical connections to one or more remote computers, such as remote computer(s) 944. The remote computer(s) 944 can be a personal computer, a server, a router, a network PC, a workstation, a microprocessor based appliance, a peer device or other common network node and the like, and typically includes many or all of the elements described relative to computer 912. For purposes of brevity, only a memory storage device 946 is illustrated with remote computer(s) 944. Remote computer(s) 944 is logically connected to computer 912 through a network interface 948 and then physically connected via communication connection 950. Network interface 948 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 950 refers to the hardware/software employed to connect the network interface 948 to the bus 918. While communication connection 950 is shown for illustrative clarity inside computer 912, it can also be external to computer 912. The hardware/software necessary for connection to the network interface 948 includes, for exemplary purposes only, internal and external technologies such as, modems including regular telephone grade modems, cable modems and DSL modems, ISDN adapters, and Ethernet cards.

FIG. 10 is a schematic block diagram of a sample-computing environment 1000 with which the subject matter of this disclosure can interact. The system 1000 includes one or more client(s) 1010. The client(s) 1010 can be hardware and/or software (e.g., threads, processes, computing devices). The system 1000 also includes one or more server(s) 1030. Thus, system 1000 can correspond to a two-tier client server model or a multi-tier model (e.g., client, middle tier server, data server), amongst other models. The server(s) 1030 can also be hardware and/or software (e.g., threads, processes, computing devices). The servers 1030 can house threads to perform transformations by employing this disclosure, for example. One possible communication between a client 1010 and a server 1030 may be in the form of a data packet transmitted between two or more computer processes.

The system 1000 includes a communication framework 1050 that can be employed to facilitate communications between the client(s) 1010 and the server(s) 1030. The client(s) 1010 are operatively connected to one or more client data store(s) 1020 that can be employed to store information local to the client(s) 1010. Similarly, the server(s) 1030 are operatively connected to one or more server data store(s) 1040 that can be employed to store information local to the servers 1030.

It is to be noted that aspects or features of this disclosure can be exploited in substantially any wireless telecommunication or radio technology, e.g., Wi-Fi; Bluetooth; Worldwide Interoperability for Microwave Access (WiMAX); Enhanced General Packet Radio Service (Enhanced GPRS); Third Generation Partnership Project (3GPP) Long Term Evolution (LTE); Third Generation Partnership Project 2 (3GPP2) Ultra Mobile Broadband (UMB); 3GPP Universal Mobile Telecommunication System (UMTS); High Speed Packet Access (HSPA); High Speed Downlink Packet Access (HSDPA); High Speed Uplink Packet Access (HSUPA); GSM (Global System for Mobile Communications) EDGE (Enhanced Data Rates for GSM Evolution) Radio Access Network (GERAN); UMTS Terrestrial Radio Access Network (UTRAN); LTE Advanced (LTE-A); etc. Additionally, some or all of the aspects described herein can be exploited in legacy telecommunication technologies, e.g., GSM. In addition, mobile as well non-mobile networks (e.g., the Internet, data service network such as internet protocol television (IPTV), etc.) can exploit aspects or features described herein.

While the subject matter has been described above in the general context of computer-executable instructions of a computer program that runs on a computer and/or computers, those skilled in the art will recognize that this disclosure also can or may be implemented in combination with other program modules. Generally, program modules include routines, programs, components, data structures, etc. that perform particular tasks and/or implement particular abstract data types. Moreover, those skilled in the art will appreciate that the inventive methods may be practiced with other computer system configurations, including single-processor or multiprocessor computer systems, mini-computing devices, mainframe computers, as well as personal computers, hand-held computing devices (e.g., PDA, phone), microprocessor-based or programmable consumer or industrial electronics, and the like. The illustrated aspects may also be practiced in distributed computing environments where tasks are performed by remote processing devices that are linked through a communications network. However, some, if not all aspects of this disclosure can be practiced on stand-alone computers. In a distributed computing environment, program modules may be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

In another example, respective components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

Various aspects or features described herein can be implemented as a method, apparatus, system, or article of manufacture using standard programming or engineering techniques. In addition, various aspects or features disclosed in this disclosure can be realized through program modules that implement at least one or more of the methods disclosed herein, the program modules being stored in a memory and executed by at least a processor. Other combinations of hardware and software or hardware and firmware can enable or implement aspects described herein, including a disclosed method(s). The term “article of manufacture” as used herein can encompass a computer program accessible from any computer-readable device, carrier, or storage media. For example, computer readable storage media can include but are not limited to magnetic storage devices (e.g., hard disk, floppy disk, magnetic strips . . . ), optical discs (e.g., compact disc (CD), digital versatile disc (DVD), blu-ray disc (BD) . . . ), smart cards, and flash memory devices (e.g., card, stick, key drive . . . ), or the like.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor may also be implemented as a combination of computing processing units.

In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or methods herein are intended to include, without being limited to including, these and any other suitable types of memory.

It is to be appreciated and understood that components, as described with regard to a particular system or method, can include the same or similar functionality as respective components (e.g., respectively named components or similarly named components) as described with regard to other systems or methods disclosed herein.

What has been described above includes examples of systems and methods that provide advantages of this disclosure. It is, of course, not possible to describe every conceivable combination of components or methods for purposes of describing this disclosure, but one of ordinary skill in the art may recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

What is claimed is:

1. A system, comprising:
at least one memory that stores computer-executable components; and
at least one processor that executes the computer-executable components stored in the at least one memory, wherein the computer-executable components comprise:
an inversion component that performs a model inversion attack on a machine learning model, and as a result, generates synthetic input data samples corresponding to training data samples used to train the machine learning model;
an assessment component that determines one or more scope characteristics of the synthetic input data samples based on a scope assessment of the synthetic input data samples; and
a verification component that determines whether the training data samples satisfy a scope criterion for the training data samples based on the one or more scope characteristics.

2. The system of claim 1, wherein the scope criterion comprises a regulatory scope criterion associated with the machine learning model.

3. The system of claim 1, wherein the one or more scope characteristics comprise a distribution of the synthetic input data samples and wherein the scope criterion comprises a target distribution for the training data samples.

4. The system of claim 3, wherein the distribution comprises a class distribution of the synthetic input data samples and wherein the target distribution comprises a target class distribution for the training data samples.

5. The system of claim 3, wherein the distribution comprises a feature distribution of the synthetic input data samples and wherein the target distribution comprises a target feature distribution for the training data samples.

6. The system of claim 3, wherein the verification component compares scope characteristic data describing the distribution to report data describing the target distribution to verify whether the training data samples satisfy the target distribution.

7. The system of claim 6, wherein the computer-executable components further comprise:
a reception component that receives the report data from an entity in association with a submission for regulatory approval of usage of the machine learning model by the entity.

8. The system of claim 1, wherein the machine learning model corresponds to a software as a medical device.

9. The system of claim 8, wherein the one or more scope characteristics account for a diversity of the synthetic data input samples with respect to different patient subgroups.

10. The system of claim 8, wherein the synthetic data samples comprise medical images and the one or more scope characteristics account for at least one of: class diversity, class coverage, feature diversity or feature coverage.

11. A method, comprising:
performing, by a system comprising a processor, a model inversion attack on a machine learning model;
generating, by the system, synthetic input data samples corresponding to training data samples used to train the machine learning model based on the preforming;
determining, by the system, one or more scope characteristics of the synthetic input data samples based on a scope assessment of the synthetic input data samples; and
determining, by the system, whether the training data samples satisfy a scope criterion for the training data samples based on the one or more scope characteristics.

12. The method of claim 11, wherein the one or more scope characteristics comprise a distribution of the synthetic input data samples and wherein the scope criterion comprises a target distribution for the training data samples.

13. The method of claim 12, wherein the distribution comprises a class distribution of the synthetic input data samples and wherein the target distribution comprises a target class distribution for the training data samples.

14. The method of claim 12, wherein the distribution comprises a feature distribution of the synthetic input data samples and wherein the target distribution comprises a target feature distribution for the training data samples.

15. The method of claim 12, further comprising:
receiving, by the system, report data describing the target distribution from an entity in association with a submission for regulatory approval of usage of the machine learning model by the entity, and wherein the determining comprises comparing scope characteristic data describing the distribution of the synthetic input data samples to the report data.

16. The method of claim 11, wherein the machine learning model corresponds to a software as a medical device.

17. The method of claim 16, wherein the one or more scope characteristics account for a diversity of the synthetic data samples with respect to different patient subgroups.

18. The method of claim 16, wherein the synthetic data samples comprise medical images and the one or more scope characteristics account for at least one of: class diversity, class coverage, feature diversity or feature coverage.

19. A non-transitory machine-readable storage medium, comprising executable instructions that, when executed by a processor, facilitate performance of operations, comprising:
performing a model inversion attack on a machine learning model;
generating synthetic input data samples corresponding to training data samples used to train the machine learning model based on the preforming;
determining one or more scope characteristics of the synthetic input data samples based on a scope assessment of the synthetic input data samples; and
determining whether the training data samples satisfy a scope criterion for the training data samples based on the one or more scope characteristics.

20. The non-transitory machine-readable storage medium of claim 19, wherein the one or more scope characteristics comprise a distribution of the synthetic input data samples and wherein the scope criterion comprises a target distribution for the training data samples.
